(19) United States
(12) Patent Application Publication
(10) Pub. No.: US 2024/0081790 A1
(43) Pub. Date: Mar. 14, 2024

(54) SAMPLE COLLECTION DEVICE

(71) Applicant: DIANA Biotechnologies, a.s., Vestec, Czechia (CZ)

(72) Inventor: Václav NAVRÁTIL, Liberec (CZ)

(21) Appl. No.: 18/252,487

(22) PCT Filed: Nov. 10, 2021
(86) PCT No.: PCT/IB21/60418

§ 371 Date: May 10, 2023

Related U.S. Application Data
(60) Provisional application No. 63/112,442, filed on Nov. 11, 2020.

(51) Int. Cl. A61B 10/00

(52) U.S. Cl. CPC A61B 10/0051

(57) ABSTRACT

A sample collection device is provided and includes a hollow upper portion, a tubular lower portion, and at least one groove. The hollow upper portion tapers between a first open end and a second open end. The upper portion also defines a sample collection volume. The tubular lower portion extends along a longitudinal axis from the second open end to an open terminal end of the device. The lower portion further defines a lumen in fluid communication with the sample collection volume and the open terminal end. The at least one groove protrudes inward from an outer surface of the lower portion and extends along at least a portion of a length of the lower portion.

### CROSS-REFERENCE TO RELATED APPLICATIONS

This application claims the benefit of U.S. Provisional Patent Application No. 63/112,442, filed on Nov. 11, 2021, and entitled “Sample Collection Device,” the entirety of which is incorporated by reference.

### BACKGROUND

To control epidemics such as influenza, severe acute respiratory syndrome (SARS), or coronavirus (e.g., COVID-19), it is desirable to perform sensitive and high throughput tests from saliva. To enable high throughput, saliva collection devices should be compatible with the format of sample vials in which polymerase chain reaction (PCR) tests are performed. This would enable the use of automated liquid handling instruments to transfer the samples directly from the primary tubes, eliminating manual work and making the testing process much faster and safer. However, existing sample collection devices are not suitable for collection of viscous liquids (e.g., saliva) and/or transfer of collected viscous liquids to automation compatible sample vials.

### SUMMARY

As an example, vials compatible with the 96-well Society for Bimolecular Screening (SBS) format have an inner diameter of about 8 mm or less. In general, it can be difficult for patients to spit into these vials. Thus, saliva is not directly collected with these vials. Furthermore, existing saliva collection tubes are bulky and are not compatible with automated liquid handling machines (e.g., 96-well SBS format). As a result, collected saliva must be transferred from such collection tubes to tubes compatible with the 96-well SBS format. Additionally, the collection volume of such large collection tubes is 1 or 2 mL. This is not suited for sample collection from older patients, as they do not create such an amount of saliva. Accordingly, there exists a need for improved devices for liquid sample collection (e.g., saliva).

Embodiments of the present disclosure relate to devices for collection of viscous liquids into small sized containers. In non-limiting embodiments, the sample collection devices can be used for collection of saliva into tubes compatible with predetermined sample vial format for automation (e.g., 96-well SBS format).

In an embodiment, a sample collection device is provided and includes a hollow upper portion, a tubular lower portion, and at least one groove. The hollow upper portion tapers between a first open end and a second open end. The upper portion also defines a sample collection volume. The tubular lower portion extends along a longitudinal axis from the second open end to an open terminal end of the device. The lower portion further defines a lumen in fluid communication with the sample collection volume and the open terminal end. The at least one groove protrudes inward from an outer surface of the lower portion and extends along at least a portion of a length of the lower portion.

In an embodiment, the hollow upper portion has a conical shape.

In an embodiment, the at least one groove includes a plurality of circumferentially spaced grooves.

In an embodiment, the at least one groove extends along approximately the entire length of the lower portion.

In an embodiment, the at least one groove extends approximately parallel to the longitudinal axis.

In an embodiment, the depth of the groove in the radial direction is from about 0.1 mm to about 1 mm.

In an embodiment, the terminal end of the lower portion is beveled.

In an embodiment, a sample collection kit is provided. The kit includes the sample collection device. The kit further includes a sample tube configured to couple to the sample collection device at an open end. The kit also includes a cap configured to seal the open end of the sample tube.

In an embodiment, the sample collection device further includes first threads positioned on an outer surface or an inner surface of the upper portion at about the second end, and the sample tube further includes second threads positioned on an inner surface or an outer surface at about the open end. The first and second threads are configured to mate with one another to couple the sample collection device to the sample tube.

In an embodiment, a length of the lower portion is dimensioned such that, when the sample collection device is coupled to the sample tube, the terminal end of the lower portion is distanced about 5 mm to about 20 mm from a base of the sample tube.

In an embodiment, an outer diameter of the lower portion is approximately equal to an inner diameter of the sample tube.

In an embodiment, a total volume of the sample tube is within the range from about 0.5 mL to about 2.0 mL.

In an embodiment, at least one of the sample collection device or the sample vial is coated with a chemical substance.

In an embodiment, the chemical substance is selected from the group consisting of chelating agents, non-ionic or ionic detergents, proteins, enzymes, proteases, RNAse inhibitors, nucleic acids, buffers, salts, or chaotropic salts.

In an embodiment, the kit can further include further at least one identifier positioned on the sample tube, where the at least one identifier encodes information identifying a sample received within the sample tube.

In an embodiment, the at least one identifier can include a first identifier positioned on a base of the sample tube.

In an embodiment, the at least one identifier can include a second identifier positioned on a sidewall of the sample tube.

In an embodiment, the at least one identifier can include at least one of a barcode, alphanumeric text, or symbol.

In another embodiment, each of the at least one identifier encodes the same information.

In another embodiment, the at least one identifier can include a foreground on a background, wherein the foreground can be a first shade, tint, or color and the background can be a second shade, tint, or color different from the first shade, tint, or color.

In an embodiment, a method of collecting a saliva sample from a patient is provided. The method includes positioning a sample collection device adjacent to a lower lip of a patient. The sample collection device includes a hollow upper portion, a tubular lower portion, and at least one groove. The hollow upper portion tapers between a first open end and a second open end and defines a sample collection volume. The tubular lower portion extends along a longitudinal axis from the second open end to an open terminal end of the device. The lower portion also defines a lumen in fluid communication with the sample collection volume and the open terminal end. The at least one groove protrudes inward from an outer surface of the lower portion and extends along at least a portion of a length of the lower portion. The method also includes receiving, within the sample collection volume, a saliva sample. The method further includes receiving, within a sample tube coupled to the sample collection device, at least a portion of the saliva sample. The method also includes decoupling the sample tube from the sample collection device after receipt of the saliva sample. The method additionally includes sealing the sample tube.

In an embodiment, the hollow upper portion has a conical shape.

In an embodiment, the at least one groove includes a plurality of circumferentially spaced grooves.

In an embodiment, the at least one groove extends along approximately the entire length of the lower portion.

In an embodiment, the at least one groove is approximately parallel to the longitudinal axis.

In an embodiment, the depth of the groove in the radial direction is from about 0.1 mm to about 1 mm.

In an embodiment, the terminal end of the lower portion is beveled.

In an embodiment, the method further includes coupling the sample collection device to the sample tube prior to receipt of the saliva sample.

In an embodiment, coupling the sample collection device to the sample tube includes engaging first threads positioned on an outer surface or an inner surface of the upper portion at about the second end with mating second threads positioned on an inner surface or an outer surface of the sample tube.

In an embodiment, the terminal end of the lower portion is distanced about 5 mm to about 20 mm from a base of the sample tube when the sample collection device is coupled to the sample tube.

In an embodiment, an outer diameter of the lower portion is approximately equal to an inner diameter of the sample tube.

In an embodiment, a total volume of the sample tube is within the range from about mL to about 2.0 mL.

In an embodiment, at least one of the sample collection device or the sample collection device is coated with a chemical substance.

In an embodiment, the chemical substance is selected from the group consisting of chelating agents, non-ionic or ionic detergents, proteins, enzymes, proteases, RNAse inhibitors, nucleic acids, buffers, salts, or chaotropic salts.

In an embodiment, the kit can further include further at least one identifier positioned on the sample tube, where the at least one identifier encodes information identifying the saliva sample.

In an embodiment, the at least one identifier can include a first identifier positioned on a base of the sample tube.

In an embodiment, the at least one identifier can include a second identifier positioned on a sidewall of the sample tube.

In an embodiment, the at least one identifier can include at least one of a barcode, alphanumeric text, or symbol.

In another embodiment, each of the at least one identifier encodes the same information.

In another embodiment, the at least one identifier can include a foreground on a background, wherein the foreground can be a first shade, tint, or color and the background can be a second shade, tint, or color different from the first shade, tint, or color.

In another embodiment, the method further includes retrieving the encoded information from the at least one identifier and decoding the encoded information to identify the saliva sample.

### BRIEF DESCRIPTION OF THE DRAWINGS

These and other features will be more readily understood from the following detailed description taken in conjunction with the accompanying drawings, in which:

FIG. 1A illustrates one exemplary embodiment of sample vials compatible with automation (e.g., 96-well SBS format). The sample vials include a sample tube having threads and screw cap configured to couple with the threads and seal the sample tube;

FIG. 1B is an isometric view of one exemplary embodiment of a sample rack containing the sample tubes of FIG. 1A;

FIG. 1C is a bottom-up view of the sample rack of FIG. 1B including optional 2D barcodes positioned on a base of the sample tubes;

FIG. 2A is a bottom-up isometric view of one exemplary embodiment of a sample collection device including upper and lower portions and at least one longitudinal groove positioned on an outer surface of the lower portion;

FIG. 2B is a top-down isometric view of the sample collection device of FIG. 2A;

FIG. 2C is an isometric view of a terminal end of the sample collection device of FIG. 2A;

FIG. 2D is a bottom view of the terminal end of the sample collection device of FIG. 2A illustrating one or more grooves formed in the lower portion;

FIG. 2E is a bottom view of an alternative embodiment of the sample collection device of FIG. 2A in which the groove(s) are in the form of a cutout portion;

FIG. 2F is a side cross-sectional view of an alternative embodiment of the sample collection device of FIG. 2A and the sample tube;

FIG. 2G is a side cross-sectional view of a further alternative embodiment of the sample collection device of FIG. 2A and the sample tube;

FIG. 3 is a bottom view of the terminal end of an alternative embodiment of the sample collection device of FIG. 2A without grooves formed in the lower portion;

FIG. 4A is a side cross-sectional view of the sample collection device of FIG. 2A with the lower portion inserted within a sample tube. A terminal end of the lower portion is spaced from a base of the sample tube by a predetermined distance D;

FIG. 4B is a side cross-sectional view of the sample collection device of FIG. 2A with the lower portion inserted within a sample tube. A terminal end of the lower portion is beveled;

FIG. 5A is one exemplary embodiment of a sample collection kit including the sample collection device of FIG. 2A, a sample tube coupled to the sample collection device, and a cap configured to seal the sample tube;

FIG. 5B is another exemplary embodiment of a sample collection kit including the sample collection device of FIG. 2A, a sample tube configured to couple to the sample collection device, and a cap sealing an open end of the sample tube;

FIG. 6 compares the form factor of the sample collection device of FIG. 2A with existing sample collection devices; and

FIG. 7 is a flow diagram illustrating one exemplary embodiment of a method for sample collection employing the sample collection device of FIG. 2A.

It is noted that the drawings are not necessarily to scale. The drawings are intended to depict only typical aspects of the subject matter disclosed herein, and therefore should not be considered as limiting the scope of the disclosure.

### DETAILED DESCRIPTION

Embodiments of sample collection devices, kits including the sample collection devices, and corresponding methods of use are presented herein. As discussed in greater detail below, the sample collection devices are configured for collection of liquids (e.g., viscous liquids) into small sized containers. In non-limiting embodiments, the sample collection devices can be used for collection of saliva into tubes compatible with predetermined sample tube formats for automation (e.g., 96-well SBS format). However, it can be understood that the disclosed embodiments can be employed for collection of other fluids without limit, such as mucus, sputum (combinations of saliva and mucus), etc.

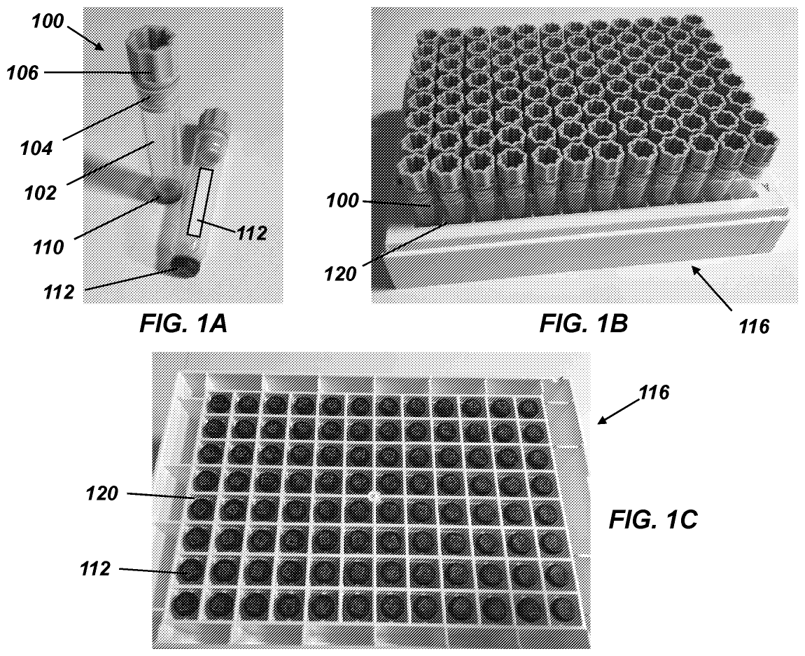

FIG. 1A illustrates one exemplary embodiment of a sample vial 100 compatible with automated liquid handing machines (e.g., for preparation of PCR testing plates). The sample vial 100 includes a sample tube 102 and a cap 106 configured to seal an open end of the sample tube 102. As shown, the sample tube 102 includes threads 104 positioned adjacent to an open end and the cap 106 includes mating threads configured to couple with the threads 104. As discussed in greater detail below, the threads 104 can be positioned on an outer surface or an inner surface of the sample tube 102. In another embodiment, not shown, the sample tube can omit the threads and the cap can be a push cap dimensioned for insertion within the open end of the sample tube to form a seal by an interference fit, also referred to as a press fit or friction fit.

Each sample vial 100 can further include at least one identifier 112 positioned thereon and including information identifying a sample contained in the sample vial 100. Embodiments of the at least one identifier 112 can include, but are not limited to, barcodes, codes/alphanumeric text/symbols, and combinations thereof. Examples of barcodes can include any barcode form, including but not limited to, 1-dimensional barcodes and 2-dimensional barcodes. In further embodiments, codes/alphanumeric text/symbols can be human-readable and/or machine-readable.

In certain embodiments, information encoded by the barcodes, other codes, alphanumeric text, symbols, or combinations thereof can be read and decoded by a machine, human, or combinations thereof to identify a sample (e.g., a saliva sample) received within the sample vial 100 (e.g., sample tube 102). Examples of such machines can include, but are not limited to, portable computing devices (e.g., smartphones, tablets, laptops) or dedicated scanning devices (e.g., including an optical scanner and processor). Accordingly, a sample contained in a sample vial 100 can be identified by machine and/or human decoding.

In further embodiments, the at least one identifier 112 can be configured to facilitate readability by machine (e.g., a barcode reader) and/or human vision. That is, the at least one identifier 112 can exhibit high contrast between a foreground and background. As an example the foreground can be a first shade, tint, or color and the background can be a second shade, tint, or color different from the first shade, tint, or color. That is, the foreground and background can be selected from contrasting shades (e.g., black, white), contrasting tints (e.g., gray), and/or contrasting colors, in any combination. In one example, the foreground can be white and the background can be black or gray. In another example, the foreground can be black or gray and the background can be white or translucent. It can be appreciated that the above-discussed examples of foreground and background are presented for illustration and that other shades, tints, and/or colors can be employed without limit.

Embodiments of the at least one identifier 112 can be formed in a variety of ways. In one aspect, the at least one identifier 112 can be formed on a label that can be secured to the sample vial 100 (e.g., an adhesive label). In another aspect, the at least one identifier 112 can be formed directly on the sample vial 100 by addition of material, removal of material, or chemical reaction. Examples can include, but are not limited to, printing (e.g., laser printing, thermal printing, dot matrix printing, etc.) one or more ink layers of a foreground and/or background, etching a foreground on a background (e.g., formation of raised markings on a portion of the sample vial 100 by melting via chemical reaction, laser, stamping, or other mechanisms), engraving (e.g., formation of recessed markings on a portion of the sample vial 100 by removal of material via a cutting tool, laser, or other mechanism), marking (e.g., laser-induced chemical reaction [oxidation] with a portion of the sample vial 100). It can be appreciated that the above-discussed techniques for forming the at least one identifier are presented for example only and that other techniques can be employed without limit.

The at least one identifier 112 can be positioned on the sample vial 100 at one or more locations, as desired. Examples of mounting locations can include, but are not limited to, the sample tube 102 (e.g., the base 110, a sidewall), the cap 106, alone or in any combination. In certain embodiments, when two or more identifiers 112 are positioned on the sample vial 100, the respective identifiers 112 can be the same type or combinations of different types (e.g., any combination of 1-dimensional barcode, 2-dimensional barcode, or human readable code/alphanumeric text/symbol). In one example, a first identifier can be a 2-dimensional barcode positioned on the base 110 of the sample tube 102 and a second identifier can be a 1-dimensional barcode or human readable code/alphanumeric text/symbol positioned on an exterior sidewall of the sample tube 102.

In further embodiments, when two or more identifiers 112 are positioned on the sample vial 100, the information encoded on each identifier 112 can be the same (e.g., an identification of the sample within the sample vial 100). Beneficially, in this manner, a user can obtain the same information regardless of which of the two or more identifiers 112 the information is retrieved from. However, it can be appreciated in alternative embodiments, the information encoded on at least two identifiers can be different.

In an example, the sample vial 100 can conform to the 96-well SBS format. FIGS. 1B-1C are diagrams illustrating top-down and bottom-up isometric views, respectively, of multiple sample vials 100 placed within a 96 SBS rack 116. As shown, each sample vial 100 is positioned within a respective well 120. The total volume of the 96-well SBS format compatible sample vial 100 can be within the range from about 0.5 mL to about 2.0 mL.

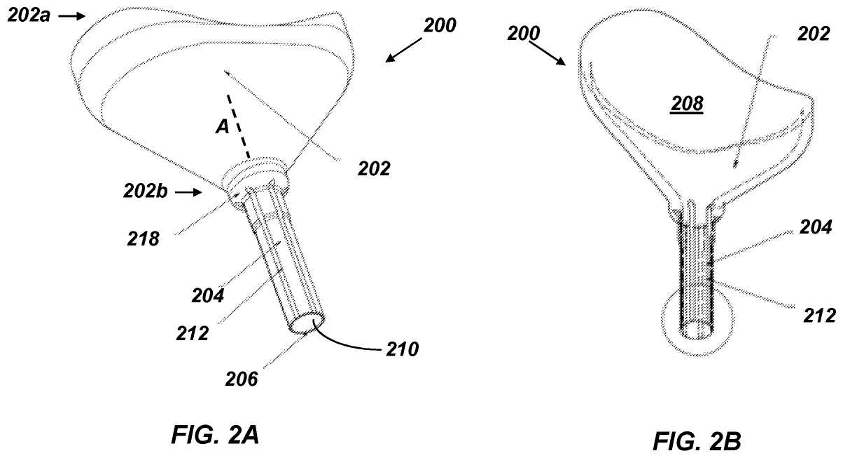

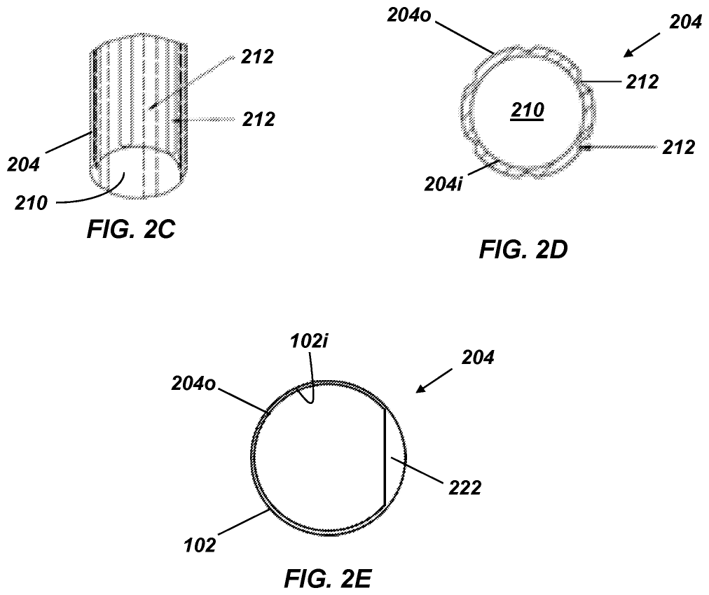

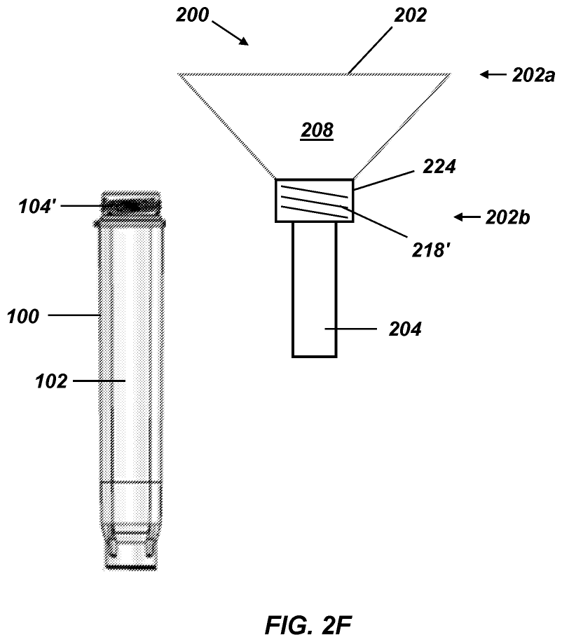

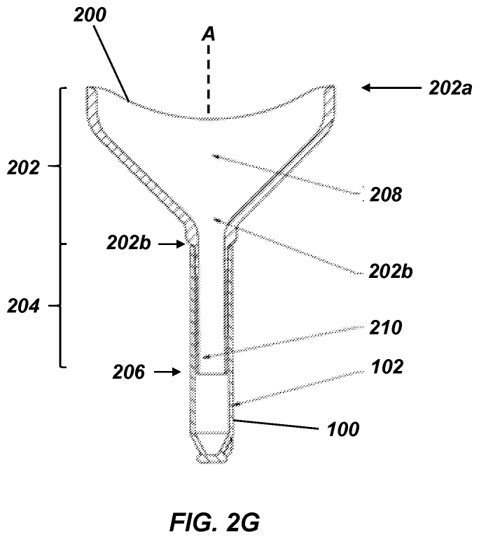

FIGS. 2A-2E are diagrams illustrating exemplary embodiments of a sample collection device 200 of the present disclosure. FIG. 2A presents a bottom-up isometric view and FIG. 2B is a top-down isometric view. As shown, the sample collection device 200 includes an upper portion 202 and a lower portion 204. The upper portion 202 is hollow and tapers between a first open end 202a and a second open end 202b, defining a sample collection volume 208 therein. In certain embodiments, the upper portion can be formed in a conical shape. The lower portion 204 is tubular and extends in the direction of a longitudinal axis A from the second open end 202b to an open terminal end 206. The lower portion 204 further defines a lumen 210 in fluid communication with the sample collection volume 208 and the open terminal end 206. It can be understood that, while the lower portion 204 is illustrated with a generally circular cross-section in FIGS. 2A-2D, alternative embodiments of the lower portion can adopt other cross-sectional shapes without limit.

In use, the sample collection device 200 is configured to couple to the sample tube 102. In one exemplary embodiment, the sample collection device 200 includes first threads 218 positioned on an outer surface of the upper portion 202. The sample tube 102 includes mating second threads (e.g., threads 104 positioned at about an open end of the sample tube 102), allowing the sample collection device 200 to be screwed onto the sample tube 102. As shown, the threads 218 are positioned at about the second open end 202b. However, in alternative embodiments (not shown), the threads can be positioned along the length of the lower portion (e.g., between the second end and the terminal end).

FIG. 2F illustrates an alternative embodiment of the sample vial 100, where the configuration of threads 104 discussed above is reversed. As an example, the sample collection device 200 includes a collar 224 at about the second open end 202b. The collar 224 extends radially outward from an exterior surface of the lower portion 204. First threads 218′ can be positioned on an interior surface of the collar 224. The sample tube 102 and mating second threads 104′ are positioned on an outer surface, adjacent to the open end of the sample tube 102.

It can be understood that the above-discussed mechanisms for coupling the sample tube and the sample collection device are merely exemplary and that other coupling mechanisms between the sample tube and the sample collection device are also envisioned. In certain embodiments, such coupling mechanisms can omit the threads on the sample tube and the sample collection device.

For example, as illustrated in FIG. 2G, an embodiment of the sample vial 100 is illustrated that includes the sample tube 102, without threads, and is configured to couple to the sample collection device 200 by an interference fit, also referred to as a press fit or friction fit. When the sample collection device 200 and the sample tube 102 are coupled, the lower portion 204 is inserted within the sample tube 102. The lower portion 204 is configured to facilitate flow of viscous fluids (e.g., saliva, mucus, sputum, etc.) from the sample collection device 200 to the sample tube 102. In one aspect, a diameter of the outer surface 204o of the lower portion 204 is approximately equal to a diameter of the inner surface 102i of the sample tube 102. This makes the lower portion 204 as wide as possible, while still permitting insertion of the lower portion 204 within the sample tube 102.

In another aspect, one or more grooves 212 are provided on an outer surface of the lower portion 204. In one embodiment, the one or more grooves 212 extend inward from the outer surface 204o of the lower portion 204, as shown in FIGS. 2A-2E. That is, the one or more grooves 212 extend inward from the outer surface 204o and terminate within the thickness of the lower portion 204 (e.g., radially outward from the inner surface 204i of the lower portion 204). In further embodiments, the configuration of the one or more grooves 212 can be varied. In one aspect, the number of the one or more grooves 212 can be varied. As shown in the cross-sectional view of FIG. 2D, six grooves 212 are present. However, in general, any number of grooves can be provided (e.g., 1, 2, 3, 4, 5, 6, 7, 8 or more) and circumferentially spaced from one another. The circumferential spacing between adjacent ones of the grooves can be approximately the same or different. In an alternative embodiment, the one or more grooves 212 can adopt the form of one or more cut-out portion 222, as illustrated in FIG. 2E.

In either case, the at least one groove 212 can extend along approximately the entire length of the lower portion 204. As an example, the at least one groove 212 can extend from about the second open end 204b of the upper portion to the open terminal end 206. In a further example (not shown), the at least one groove can be formed at a predetermined distance from the second open end and extend to the open terminal end 206.

In another aspect, the shape of the at least one groove 212 can vary. As shown, the at least one groove 212 is straight and extends approximately parallel to the longitudinal axis A. In a further example, the at least one groove can adopt a curved (e.g., wavy) shape extending in the longitudinal direction.

In a further aspect, the geometry of the at least one groove 212 can vary. As shown, the at least one groove 212 adopts an approximately hemispherical cross-section. However, in alternative embodiments, the at least one groove can adopt any desired cross-sectional shape (e.g., circular, ovular, square, triangular, etc.) The depth of the at least one groove 212 extends by a predetermined depth in the radial direction (e.g., inward). The depth can range from about 0.1 mm to about 1.0 mm.

As further shown, in one embodiment, each of the at least one groove 212 is approximately the same (e.g., same length, path, cross-sectional geometry, depth, etc.) However, in alternatively embodiments where two or more grooves are present, at least one groove can be different from other grooves.

It has been discovered that the at least one groove 212 provides improved fluid flow, as compared to other configurations. Consider one example where the diameter of the outer surface 204o of the lower portion 204 is approximately equal to the diameter of an inner surface 102i of the sample tube 102 and the at least one groove 212 is/are absent. That is, the inner surface 102i is very close to, or contacts, the outer surface 204o of the lower portion 204. It has been observed that saliva received within the upper portion 202 does not enter the sample tube 102.

Without being bound by theory, it is believed that the at least one groove 212 facilitates fluid flow from the sample collection device 200 (e.g., the lower portion 204) to the sample tube 102 by providing a pathway for air to flow out of the sample tube 102. In order for fluid to flow from the lower portion 204 to the sample tube 102, air ahead of the liquid is displaced from the lower portion 204 to the sample tube 102. Absent the at least one groove 212, air flow out of the sample tube 102 is impeded, resulting in an increase in air pressure within the sample tube 102 due to the displaced air and resistance to fluid flow. In contrast, when the at least one groove 212 is present, air within the sample tube 102 can be displaced from the sample tube 102 via the at least one groove 212, avoiding pressure buildup and attendant resistance to fluid flow into the sample tube 102.

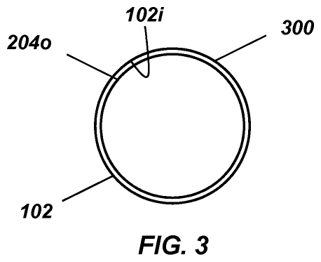

Consider another example where the diameter of the outer surface 204o of the lower portion 204 is less than the diameter of the inner surface 102i of the sample tube 102 and the at least one groove 212 is/are absent, as shown in FIG. 3. This configuration forms a space 300 between the outer surface 204o of the lower portion 204 and the inner surface 102i of the sample tube 102 which allows for air to exit the sample tube 102. However, it has been observed that saliva received within the upper portion 202 either does not enter the lower portion 204, or enters the lower portion 204 but does not enter the sample tube 102. Without being bound by theory, it is believed that the reduction in diameter (e.g., inner diameter) of the lower portion 204 inhibits fluid flow from the lower portion 204 to the sample tube 102.

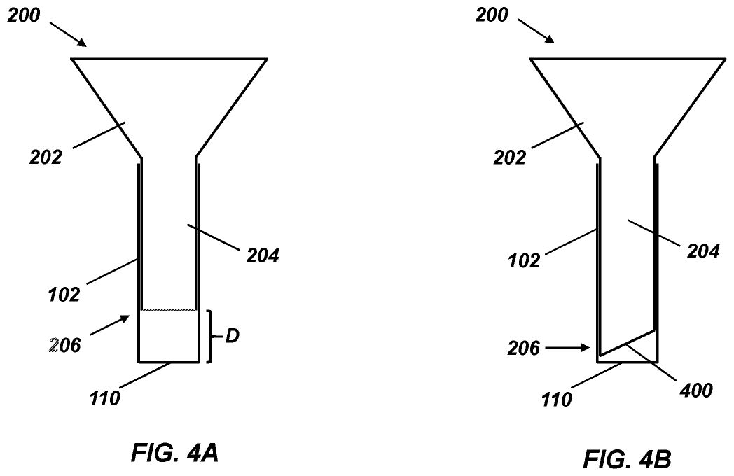

In further embodiments of the sample collection device 200, the length of the lower portion 204 can be varied. As shown in FIG. 4A, the length of the lower portion 204 can be dimensioned such that the open terminal end 206 is positioned at a predetermined distance D from the base 110 of the sample tube 102. In certain embodiments, the distance D can be within the range from about 5 mm to about 20 mm. In alternative embodiments, the length of the lower portion 204 can be further reduced, such that the distance D is greater than 10 mm, or the length of the lower portion 204 can be further increased such that the distance D is less than 5 mm.

In other embodiments of the sample collection device 200, the shape of the open terminal end 206 can be varied. In one embodiment, as shown in FIGS. 2A-2E, the open terminal end 206 can be approximately flat (e.g., horizontal) and oriented approximately perpendicular to the longitudinal axis A and the sidewalls of the lower portion 204. However, in an alternative embodiments, shown in FIG. 4B, the open terminal end 206 can include a bevel 400.

In further embodiments, at least one of the sample tube 100 or the sample collection device 200 can be coated with a chemical substance. The chemical substance is configured to ease handling of collected viscous fluids and/or stabilize biological molecules in collected viscous fluids, facilitating sample collection and shipping at ambient temperature.

In an embodiment, the coating can be applied to interior surfaces the sample vial 100 and/or the sample collection device 200 that are expected to contact the collected viscous fluids. In certain embodiments, the chemical substance adheres to the sample vial 100 and/or sample collection device 200 such that it does not leave the sample vial 100 and/or sample collection device 200 during use, and even in case of flipping or falling down, protecting the patient from inhaling or swallowing the chemical substance. In other embodiments, the coating can be a powder applied to interior surfaces of the sample vial 100 or the sample collection device 200. Examples of chemical substances suitable for the coating can include, but are not limited to, chelating agents (e.g., EDTA, EGTA, citrate), non-ionic or ionic detergents (e.g., Tween™, SDS), proteins, enzymes, proteases (e.g., protease K), RNAse inhibitors, nucleic acids, buffers, salts, or chaotropic salts (e.g., Guanidinium thiocyanate).

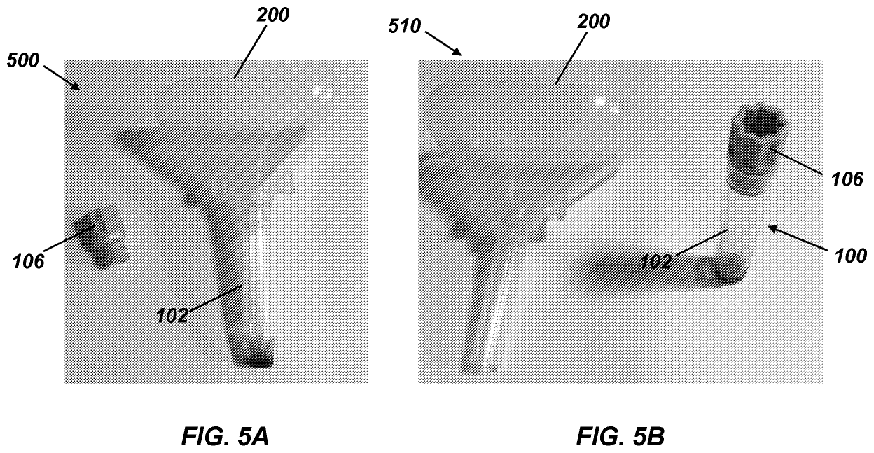

FIGS. 5A-5B illustrate embodiments of kits 500, 510 including the sample collection device 200. Each of the kits 500, 510 can include the sample collection device 200 and the sample vial 100 (sample tube 102 and cap 106). The kit 500 can be configured with the sample collection device 200 coupled to the sample tube 102, while the kit 510 can be configured with the sample tube 102 decoupled from sample collection device 200. Embodiments of kits can be further provided using the sample vial 100 and sample collection device 200 configured with the reverse thread configurations 104′, 218′ (e.g., FIG. 2F) or configured without threads (e.g., FIG. 2G).

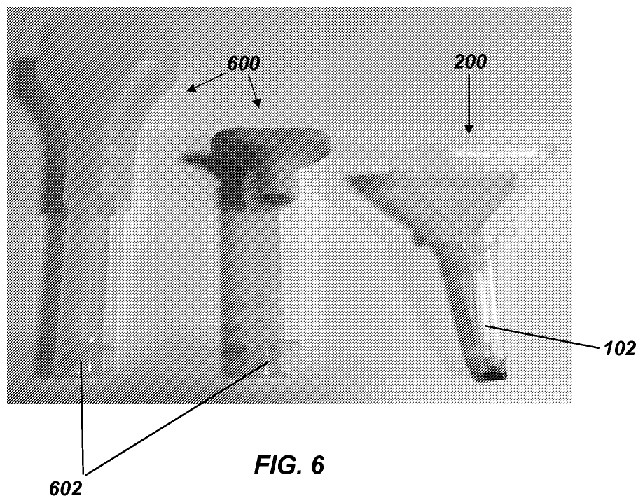

FIG. 6 compares the form factor of existing sample collection devices 600 to an embodiment of the sample collection device 200. It can be observed that the existing sample collection devices 600 are relatively bulky, as compared to the sample collection device 200. Furthermore, the size of collection tubes 602 configured for use with the existing sample collection devices 600 are not suitable for automated liquid handling machines (e.g., for preparation of PCR testing plates).

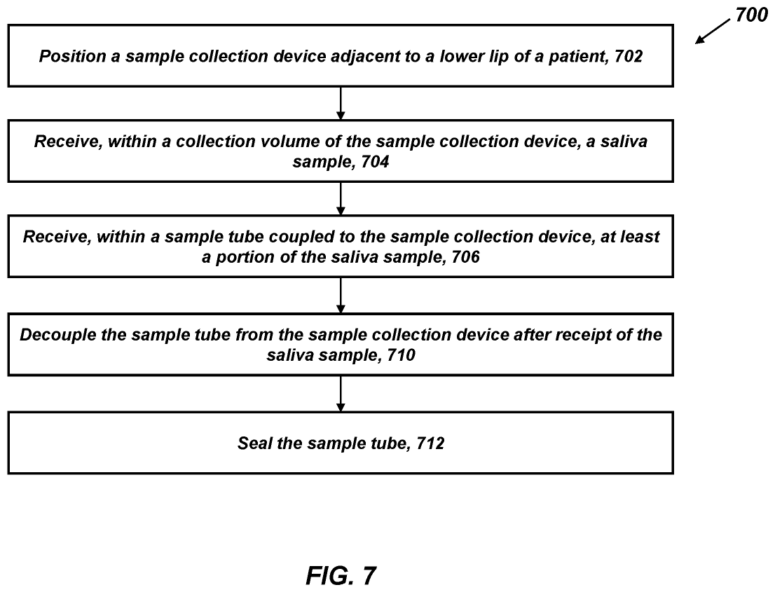

FIG. 7 is a flow diagram illustrating one exemplary embodiment of a method 700 for sample collection employing embodiments of the sample collection device 200. As shown, the method 700 includes operations 702-712. However, alternative embodiments of the method can include greater or fewer operations than illustrated in FIG. 7 and the operations can be performed in a different order than illustrated in FIG. 7.

In operation 702, a sample collection device is positioned adjacent to a lower lip of a patient. The sample collection device can be the sample collection device 200 including the upper portion 202, the lower portion 204, and the at least one groove 212. As discussed above, the upper portion 202 can taper between the first open end 202a and the second open end 202b and define the sample collection volume 208. The lower portion 204 can extend along the longitudinal axis A from the second open end 202b to the open terminal end 206. The lower portion 204 can also define the lumen 210, which is in fluid communication with the sample collection volume 208 and the open terminal end 206. The at least one groove 212 protrudes inward from the outer surface 204o of the lower portion 204 and extends along at least a portion of a length of the lower portion 204.

In operation 704, a viscous fluid sample (e.g., saliva, mucus, sputum, etc.) is received within the sample collection volume 208. As an example, the patient can spit into the upper portion 202.

In operation 706, at least a portion of the saliva sample is received within the sample vial 100 (e.g., sample tube 102) coupled to the sample collection device 200. In certain embodiments, the sample tube 102 can be already coupled to the sample collection device 200 within the kit 500. In other embodiments, the sample tube 102 can be decoupled from the sample collection device 200 within the kit 510. Accordingly, the method 700 can also include coupling the sample tube 102 to the sample collection device 200. The saliva sample can flow from the upper portion 202, through the lower portion 204, and into the sample tube 102. As discussed above, air displaced into the sample tube 102 from the lower portion 204 can exit the sample tube 102 via the at least one groove 212.

In operations 710-712, the sample tube 102 is decoupled from the sample collection device 200 after receipt of the saliva sample and sealed (e.g., by coupling the cap 106 to the sample tube 102).

Exemplary technical effects of the methods, systems, and devices described herein include, by way of non-limiting example improved flow of sample fluids (e.g., saliva, mucus, sputum, etc.) from a patient to a sample tube. The sample tubes can be configured for use with automated liquid handling machines (e.g., for preparation of PCR testing plates), avoiding the need for time consuming transfer of collected saliva samples from sample tubes that cannot be used with automated liquid handlers.

Certain exemplary embodiments have been described to provide an overall understanding of the principles of the structure, function, manufacture, and use of the systems, devices, and methods disclosed herein. One or more examples of these embodiments have been illustrated in the accompanying drawings. Those skilled in the art will understand that the systems, devices, and methods specifically described herein and illustrated in the accompanying drawings are non-limiting exemplary embodiments and that the scope of the present invention is defined solely by the claims. The features illustrated or described in connection with one exemplary embodiment may be combined with the features of other embodiments. Such modifications and variations are intended to be included within the scope of the present invention. Further, in the present disclosure, like-named components of the embodiments generally have similar features, and thus within a particular embodiment each feature of each like-named component is not necessarily fully elaborated upon.

Approximating language, as used herein throughout the specification and claims, may be applied to modify any quantitative representation that could permissibly vary without resulting in a change in the basic function to which it is related. Accordingly, a value modified by a term or terms, such as “about,” “approximately,” and “substantially,” are not to be limited to the precise value specified. In at least some instances, the approximating language may correspond to the precision of an instrument for measuring the value. Here and throughout the specification and claims, range limitations may be combined and/or interchanged, such ranges are identified and include all the sub-ranges contained therein unless context or language indicates otherwise.

In the descriptions herein and in the claims, phrases such as “at least one of” or “one or more of” may occur followed by a conjunctive list of elements or features. The term “and/or” may also occur in a list of two or more elements or features. Unless otherwise implicitly or explicitly contradicted by the context in which it is used, such a phrase is intended to mean any of the listed elements or features individually or any of the recited elements or features in combination with any of the other recited elements or features. For example, the phrases “at least one of A and B;” “one or more of A and B;” and “A and/or B” are each intended to mean “A alone, B alone, or A and B together.” A similar interpretation is also intended for lists including three or more items. For example, the phrases “at least one of A, B, and C;” “one or more of A, B, and C;” and “A, B, and/or C” are each intended to mean “A alone, B alone, C alone, A and B together, A and C together, B and C together, or A and B and C together.” In addition, use of the term “based on,” above and in the claims is intended to mean, “based at least in part on,” such that an unrecited feature or element is also permissible.

It is understood that where a parameter range is provided, all integers within that range, and tenths thereof, are also provided by embodiments of the disclosure. For example, “0.2-5 mg” is a disclosure of 0.2 mg, 0.3 mg, 0.4 mg, 0.5 mg, 0.6 mg etc. up to and including 5.0 mg.

As used in the description herein and throughout the claims that follow, the meaning of “a,” “an,” and “the” includes plural reference unless the context clearly dictates otherwise.

One skilled in the art will appreciate further features and advantages of the invention based on the above-described embodiments. Accordingly, the present application is not to be limited by what has been particularly shown and described, except as indicated by the appended claims. All publications and references cited herein are expressly incorporated by reference in their entirety.

1. A sample collection device, comprising:
a hollow upper portion that tapers between a first open end and a second open end, the upper portion defining a sample collection volume;
a tubular lower portion extending along a longitudinal axis from the second open end to an open terminal end of the device, the lower portion defining a lumen in fluid communication with the sample collection volume and the open terminal end; and
at least one groove extending inward from an outer surface of the lower portion, the at least one groove extending along at least a portion of a length of the lower portion.

2. The device of claim 1, wherein the hollow upper portion has a conical shape.

3. The device of claim 1, wherein the at least one groove comprises a plurality of circumferentially spaced grooves.

4. The device of claim 1, wherein the at least one groove extends along approximately the entire length of the lower portion.

5. The device of claim 1, wherein the at least one groove extends approximately parallel to the longitudinal axis.

6. The device of claim 1, wherein the depth of the groove in the radial direction is from about 0.1 mm to about 1.0 mm.

7. The device of claim 1, wherein the terminal end of the lower portion is beveled.

8. A sample collection kit comprising:
the sample collection device of claim 1;
a sample tube configured to couple to the sample collection device at an open end; and
a cap configured to seal the open end of the sample tube.

9. The kit of claim 8, wherein the sample collection device further comprises first threads positioned on an outer surface or inner surface of the upper portion at about the second end, and the sample tube further comprises second threads positioned on an inner surface or an outer surface at about the open end, wherein the first and second threads are configured to mate with one another to couple the sample collection device to the sample tube.

10. The kit of claim 8, wherein a length of the lower portion is dimensioned such that, when the sample collection device is coupled to the sample tube, the terminal end of the lower portion is distanced about 5 mm to about 20 mm from a base of the sample tube.

11. The kit of claim 8, wherein an outer diameter of the lower portion is approximately equal to an inner diameter of the sample tube.

12. The kit of claim 8, wherein a total volume of the sample tube is within the range from about 0.5 mL to about 2.0 mL.

13. The kit of claim 8, wherein at least one of the sample collection device or the sample vial is coated with a chemical substance.

14. The kit of claim 13, wherein the chemical substance is selected from the group consisting of chelating agents, non-ionic or ionic detergents, proteins, enzymes, proteases, RNAse inhibitors, nucleic acids, buffers, salts, or chaotropic salts.

15. The kit of claim 8, further comprising at least one identifier positioned on the sample tube, wherein the at least one identifier encodes information identifying a sample received within the sample tube.

16. The kit of claim 15, wherein the at least one identifier comprises a first identifier positioned on a base of the sample tube.

17. The kit of claim 15, wherein the at least one identifier comprises a second identifier positioned on a sidewall of the sample tube.

18. The kit of claim 15, wherein the at least one identifier comprises at least one of a barcode, alphanumeric text, or symbol.

19. The kit of claim 15, wherein each of the at least one identifier encodes the same information.

20. The kit of claim 15, wherein the at least one identifier comprises a foreground on a background, wherein the foreground is a first shade, tint, or color and the background is a second shade, tint, or color different from the first shade, tint, or color.

21. A method of collecting a liquid sample from a patient, comprising:
positioning a sample collection device adjacent to a lower lip of a patient, the sample collection device comprising:
a hollow upper portion that tapers between a first open end and a second open end, the upper portion defining a sample collection volume;
a tubular lower portion extending along a longitudinal axis from the second open end to an open terminal end of the device, the lower portion defining a lumen in fluid communication with the sample collection volume and the open terminal end; and
at least one groove extending inward from an outer surface of the lower portion, the at least one groove extending along at least a portion of a length of the lower portion;
receiving, within the sample collection volume, a saliva sample;
receiving, within a sample tube coupled to the sample collection device, at least a portion of the saliva sample;
decoupling the sample tube from the sample collection device after receipt of the saliva sample; and
sealing the sample tube.

22. The device of claim 21, wherein the hollow upper portion has a conical shape.

23. The method of claim 21, wherein the at least one groove comprises a plurality of circumferentially spaced grooves.

24. The method of claim 21, wherein the at least one groove extends along approximately the entire length of the lower portion.

25. The method of claim 21, wherein the at least one groove is approximately parallel to the longitudinal axis.

26. The method of claim 21, wherein the depth of the groove in the radial direction is from about 0.1 mm to about 1.0 mm.

27. The method of claim 21, wherein the terminal end of the lower portion is beveled.

28. The method of claim 21, further comprising coupling the sample collection device to the sample tube prior to receipt of the saliva sample.

29. The method of claim 28, wherein coupling the sample collection device to the sample tube comprises engaging first threads positioned on an outer surface of the upper portion at about the second end with mating second threads positioned on an inner surface of the sample tube.

30. The method of claim 21, wherein the terminal end of the lower portion is distanced about 5 mm to about 20 mm from a base of the sample tube when the sample collection device is coupled to the sample tube.

31. The method of claim 21, wherein an outer diameter of the lower portion is approximately equal to an inner diameter of the sample tube.

32. The method of claim 21, wherein a total volume of the sample tube is within the range from about 0.5 mL to about 2.0 mL.

33. The method of claim 21, wherein at least one of the sample collection device or the sample collection device is coated with a chemical substance.

34. The method of claim 21, wherein the chemical substance is selected from the group consisting of chelating agents, non-ionic or ionic detergents, proteins, enzymes, proteases, RNAse inhibitors, nucleic acids, buffers, salts, or chaotropic salts.

35. The method of claim 21, further comprising at least one identifier positioned on the sample tube, the at least one identifier encoding information identifying the saliva sample.

36. The method of claim 35, wherein the at least one identifier comprises a first identifier positioned on a base of the sample tube.

37. The method of claim 35, wherein the at least one identifier comprises a second identifier positioned on a sidewall of the sample tube.

38. The method of claim 35, wherein the at least one identifier comprises at least one of a barcode, alphanumeric text or symbol.

39. The method of claim 35, wherein each of the at least one identifier encodes the same information.

40. The method of claim 35, wherein the at least one identifier comprises a foreground on a background, wherein the foreground is a first shade, tint, or color and the background is a second shade, tint, or color different from the first shade, tint, or color.

41. The method of claim 35, further comprising retrieving the encoded information from the at least one identifier and decoding the encoded information to identify the saliva sample.
